(19) United States
(12) Patent Application Publication
(10) Pub. No.: US 2024/0172960 A1
(43) Pub. Date: May 30, 2024

(54) METHOD AND APPARATUS FOR USING AN EXPANDABLE ELEMENT TO ASCERTAIN VESSEL DIAMETER

(71) Applicant: Boston Scientific Scimed, Inc., Maple Grove, MN (US)

(72) Inventors: Paul F. Chouinard, Maple Grove, MN (US); Brady Scott Logan, Monticello, MN (US); Ewnet Gebrehiwot, Eden Prairie, MN (US)

(73) Assignee: Boston Scientific Scimed, Inc., Maple Grove, MN (US)

(21) Appl. No.: 18/521,617

(22) Filed: Nov. 28, 2023

Related U.S. Application Data
(60) Provisional application No. 63/428,629, filed on Nov. 29, 2022.

(51) Int. Cl. A61B 5/107; A61B 5/02

(52) U.S. Cl. CPC A61B 5/1076; A61B 5/02007

(57) ABSTRACT

An effective vessel diameter of a blood vessel at a deployment site may be determined using a measurement apparatus that includes an expandable element that is moveable between a collapsed configuration for advancement and an expanded configuration for measurement. The measurement apparatus may be advanced to the deployment site with the expandable element in its collapsed configuration. The expandable element may be caused to expand from its collapsed configuration to its expanded configuration and one or more measurements pertaining to the expandable element in its expanded configuration may be obtained. The one or more measurements may be used to determine an effective vessel diameter at the deployment site.

### CROSS-REFERENCE TO RELATED APPLICATIONS

This application claims the benefit of priority under 35 U.S.C. § 119 of U.S. Provisional Application No. 63/428,629, filed Nov. 29, 2022, the entire disclosure of which is hereby incorporated by reference.

### TECHNICAL FIELD

The disclosure relates generally to medical devices and more particularly to medical devices that are adapted for use in ascertaining vessel diameter for optimal stent selection.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a method of determining an effective vessel diameter at a deployment site within a vessel using a measurement apparatus, the measurement apparatus including an expandable element that is moveable between a collapsed configuration for advancement and an expanded configuration for measurement. The method includes advancing the measurement apparatus to the deployment site with the expandable element in its collapsed configuration and causing the expandable element to expand from its collapsed configuration to its expanded configuration. One or more measurements pertaining to the expandable element in its expanded configuration are obtained. The obtained one or more measurements are used to determine an effective vessel diameter at the deployment site.

Alternatively or additionally, the expandable element may be adapted to limit its expanded configuration when the expandable element apposes an interior wall of the vessel.

Alternatively or additionally, the measurement apparatus may include an outer sheath adapted to extend over the expandable element, and causing the expandable element to expand from its collapsed configuration to its expanded configuration may include withdrawing the outer sheath proximally to uncover the expandable element.

Alternatively or additionally, the expandable element may be adapted to regain its expanded configuration when no longer covered by the outer sheath.

Alternatively or additionally, the measurement apparatus may include an outer member secured to a proximal region of the expandable element and an inner member extending through an interior of the outer member, the inner member secured to a distal region of the expandable element Causing the expandable element to expand from its collapsed configuration to its expanded configuration may include withdrawing the inner member proximally in order to shorten a length of the expandable element, thereby causing the expandable element to move towards its expanded configuration.

Alternatively or additionally, obtaining one or more measurements may include using fluoroscopy to determine a diameter of the expandable element in its expanded configuration.

Alternatively or additionally, obtaining one or more measurements may include determining one or more changes for the expandable element in moving from its collapsed configuration to its expanded configuration.

Another example may be found in an apparatus for determining an effective diameter of a vessel. The apparatus includes an expandable element having a proximal region and a distal region, the expandable element moveable between a collapsed configuration in which the expandable element has a collapsed length and a collapsed diameter and an expanded configuration in which the expandable element has a decreased length and an increased diameter. The apparatus includes an outer elongate member having a distal region secured relative to the proximal region of the expandable element, the outer elongate member defining a lumen extending therewithin, and an inner elongate member having a distal region secured relative to the distal region of the expandable element, the inner elongate member extending through the lumen. Proximal movement of the inner elongate member relative to the outer elongate member causes the expandable element to decrease in length and thus increase in diameter until the braid urges the vessel into a more uniform cross-sectional shape. The expandable element is adapted to be visible under imaging techniques such as fluoroscopy and ultrasound such that one or more dimensions of the expandable element are measurable.

Alternatively or additionally, the one or more dimensions of the expandable element may include a diameter of the expandable element.

Alternatively or additionally, the one or more dimensions of the expandable element may include a change in length of the expandable element.

Alternatively or additionally, the apparatus may further include an indicator that provides a diameter of the expandable element.

Alternatively or additionally, the indicator may include a plurality of markings disposed along the inner elongate member.

Alternatively or additionally, the indicator may include a rotary dial secured relative to the apparatus, the rotary dial coupled with a screw drive that causes rotation of the rotary dial to cause translation of the inner elongate member relative to the outer elongate member; and wherein the indicator comprises an alphanumeric scale that shows a diameter or a length of the expandable element.

Alternatively or additionally, the expandable element may include a spiral cut hypotube.

Alternatively or additionally, the expandable element may include a braid.

Alternatively or additionally, the one or more dimensions of the expandable element may include a change in braid angle.

Another example may be found in an apparatus for determining an effective diameter of a vessel, the vessel including vessel walls. The apparatus includes a braid having a proximal region and a distal region, the braid moveable between a collapsed configuration and an expanded configuration, the braid adapted to be visible under fluoroscopy, and an outer sheath overlying the braid, the outer sheath constraining the braid in its collapsed configuration while the outer sheath overlies the braid. The outer sheath is adapted to be withdrawn proximally to expose the braid, thereby allowing the braid to expand towards its expanded configuration until braid apposes the vessel walls, the braid expansion limited by interaction with the vessel walls. An imaging technique may be used to ascertain one or more dimensions of the braid.

Alternatively or additionally, the one or more dimensions of the braid may include a diameter of the braid.

Alternatively or additionally, the one or more dimensions of the braid may include a change in length of the braid.

Alternatively or additionally, the one or more dimensions of the braid may include a change in braid angle.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative apparatus including a passively actuated expansion element;

FIG. 2 is a schematic view of an illustrative apparatus including an actively actuated expansion element;

FIG. 3 is a schematic view of an illustrative apparatus including an actively actuated expansion element shown in a collapsed configuration;

FIG. 4 is a schematic view of the illustrative apparatus of FIG. 3, with the actively actuated expansion element shown in an expanded configuration;

FIG. 5 is a schematic view of an illustrative apparatus including an actively actuated expansion element;

FIG. 6 is a schematic view of a portion of an illustrative apparatus including an actively actuated expansion element;

FIG. 7 is a schematic view of a portion of an illustrative apparatus including an actively actuated expansion element;

FIG. 7A is an enlarged schematic view of a portion of the illustrative apparatus of FIG. 7;

FIG. 8 is a schematic view of a portion of an illustrative apparatus including an actively actuated expansion element;

FIG. 9 is a schematic view of an illustrative apparatus including an actively actuated expansion element shown in a collapsed configuration; and

FIG. 10 is a schematic view of the illustrative apparatus of FIG. 9, with the actively actuated expansion element shown in an expanded configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Stents are implanted within the vasculature for a variety of reasons. In some cases, an important consideration when selecting a stent for implantation is properly sizing the stent. If the stent is too small, it may not remain in position. If the stent is too large, this can cause a variety of complications. A potential complication in determining an appropriate size for a stent is that the blood vessels such as veins in which the stent is to be deployed can have an irregular cross-sectional shape. Moreover, because the veins function as a capacitive system, the veins can change in size as a result of changes in blood pressure, for example. These factors can make stent sizing more complicated. In some cases, a vein diameter may be estimated by looking at minimum and maximum cross-sectional dimensions, and taking an average as the diameter.

In some cases, a vein diameter may be estimated by treating the vein as having a circular cross-section. For example, a vein diameter may be estimated by determining the cross-sectional area of the vein, and calculating a diameter from the cross-sectional area using the well-known relationship A=πr² (A is area, r is radius). In some cases, a vein diameter may be estimated by determining the perimeter of a cross-section and calculating a diameter from the perimeter using the well-known relationship P=2πr (P is perimeter). Each of these methods may provide a different diameter estimate for the same vein. Moreover, each of these methods may not appropriately compensate for phasic or positional changes in diameter, oblong or flattened cross-sections, or errors caused by non-perpendicular measurement planes.

In some cases, an expandable element may be disposed within a vessel to be measured, and the expandable element may be allowed to passively expand until the expandable element contacts the vessel walls and at least partially reshapes the vessel. In some cases, the expandable element may be actively expanded. In either event, the goal is to temporarily reshape the vessel in order to obtain a more accurate vessel diameter, not to excessively stretch or otherwise resize the vessel. An estimated diameter of the vessel may be determined by directly measuring the expanded diameter of the expandable element, by using imaging techniques such as ultrasound, or fluoroscopy in instances in which the expandable element is sufficiently radiopaque. An estimated diameter of the vessel may be determined indirectly, such as by measuring one or more other parameters of the expandable element. In some cases, for example when the expandable element is a braid, there may be known relationships between the diameter of the braid and the length of the braid. This means that for a given change in length, a new diameter can be ascertained. In some cases, changes in braid angle may be used to ascertain how the diameter of the braid has changed as the braid has expanded.

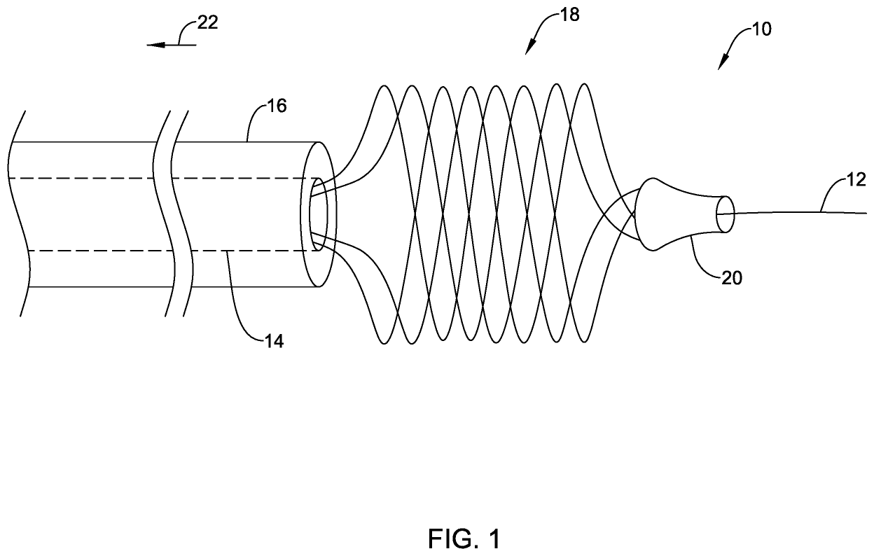

FIG. 1 is a schematic view of an illustrative apparatus 10 that may be used in determining an effective vessel diameter. In this, an effective vessel diameter may be considered as being a diameter that corresponds to the vessel having been at least partially reshaped in the process of making the measurement. The reshaping is considered to be temporary, as the vessel will revert to its native profile once the apparatus 10 has been withdrawn. The effective vessel diameter may be considered as being equivalent to a stent diameter of a stent that is an appropriate size to be deployed at that particular location. The stent should have a diameter that is sufficient to at least partially reshape the vessel when implanted, as this will facilitate the stent remaining in position until such time as tissue ingrowth has further secured the stent in place, without distending the vessel. A guidewire 12 may be advanced through the vasculature until the guidewire 12 reaches and slightly passes a desired stent deployment site. The apparatus 10 may be advanced over the guidewire 12 until the apparatus reaches the desired stent deployment site so that one or more measurements of a vessel's effective diameter may be taken.

The apparatus 10 includes an inner shaft 14 and an outer sheath 16. An expandable element 18 is secured to the inner shaft 14. In some cases, the expandable element 18 may be secured to the inner shaft 14 at a single location, such as but not limited to a proximal end of the expandable element 18. In some cases, the expandable element 18 may include a floating tip 20 that allows the distal end of the expandable element 18 to float relative to the inner shaft 14. As a result, the expandable element 18 is free to adjust its length as the expandable element 18 changes its diameter. In some cases, the expandable element 18 may be movable between a collapsed configuration and an expanded configuration (shown). In some cases, the expandable element 18 may have a remembered configuration corresponding to the expanded configuration. The outer sheath 16 may be adapted to constrain the expandable element 18 in the collapsed configuration for delivery. Once the apparatus 10 has reached a site at which a measurement is desired, the outer sheath 16 may be withdrawn proximally, in a direction indicated by an arrow 22, in order to allow the expandable element 18 to expand.

The expandable element 18 may be adapted to be biased into its remembered, or expanded configuration. In some cases, the expandable element 18 may be a braid. The expandable element 18 may be formed of any desired material adapted to exhibit a remembered configuration. In some cases, the expandable element 18 may be formed of a shape memory material. The expandable element 18 may include or be formed of a radiopaque material that is visible via fluoroscopy. The expandable element 18 may be adapted to provide an outward force against vessel walls when moving into its expanded configuration without exerting too much force on the vessel walls. In some cases, the expandable element 18 may be adapted to exert enough force on the vessel walls in order to at least partially reshape the vessel, but not so much force as to cause the vessel walls to stretch.

Once the expandable element 18 has reached an expanded configuration corresponding to a temporary partial remodeling of the vessel at the deployment site, the effective diameter of the vessel may be obtained by measuring the current diameter of the expandable element 18. In some cases, this may be done using fluoroscopy to visualize the expandable element 18 itself. Fluoroscopy allows measurements to be taken of features and elements that are visualized during fluoroscopy. In some cases, instead of measuring the current diameter of the expandable element 18, it may be easier to determine a current length of the expandable element 18. It will be appreciated that for some expandable elements 18 such as braids, there may be known relationships between changes in length of the braid and corresponding changes in diameter of the braid. In some cases, it may be feasible to determine a change in braid angle of the braid. There are known relationships between changes in braid angle, changes in diameter and changes in length. Once the effective diameter has been directly measured or indirectly inferred, the outer sheath 16 may be advanced distally over the expandable element 18 to collapse the expandable element 18 into the collapsed configuration for withdrawal or for moving to another measurement site.

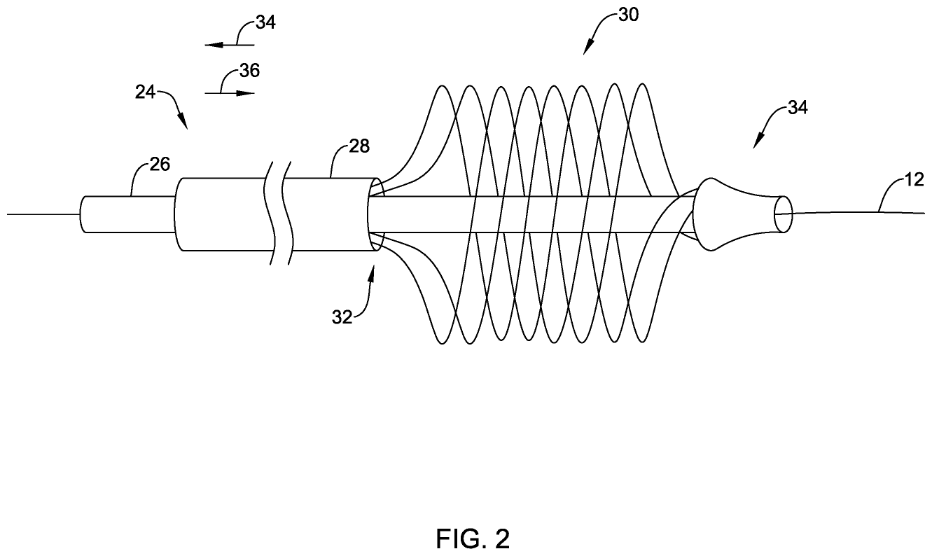

FIG. 2 is a schematic view of an illustrative apparatus 24 that may be used in determining an effective vessel diameter. In this, an effective vessel diameter may be considered as being a diameter that corresponds to the vessel having been at least partially reshaped in the process of making the measurement. The reshaping is considered to be temporary, as the vessel will revert to its native profile once the apparatus 24 has been withdrawn. The effective vessel diameter is commonly considered in the selection of a stent diameter of a stent that is an appropriate size to be deployed at that particular location. The stent should have a diameter that is sufficient to at least partially reshape the vessel when implanted, as this will facilitate the stent remaining in position until such time as tissue ingrowth has further secured the stent in place, without distending the vessel. The guidewire 12 may be advanced through the vasculature until the guidewire 12 reaches and slightly passes a desired stent deployment site. The apparatus 24 may be advanced over the guidewire 12 until the apparatus reaches the desired stent deployment site so that one or more measurements of a vessel's effective diameter may be taken.

The apparatus 24 includes an inner shaft 26 and an outer shaft 28. The inner shaft 26 is movable relative to the outer shaft 28. The apparatus 24 includes an expandable element 30 having a proximal end 32 that is secured to the outer shaft 28 and a distal end 34 that is secured to the inner shaft 26. As a result, the length of the expandable element 30 may be changed by translating the inner shaft 26 relative to the outer shaft 28. Withdrawing the inner shaft 26 proximally, in a direction indicated by an arrow 34, relative to the outer shaft 28 will cause the expandable element 30 to shorten in length. As the expandable element 30 shortens in length, the expandable element 30 will increase in diameter. Advancing the inner shaft 26 distally, in a direction indicated by an arrow 36, relative to the outer shaft 28 will cause the expandable element 30 to increase in length. As the expandable element 30 increases in length, the expandable element 30 will decrease in diameter. Accordingly, an operator is able to cause the expandable element 30 to reach an expanded configuration in which the expandable element 30 has contacted the vessel walls of the vessel in which the apparatus 24 is deployed, and the operator has control over how much the expandable element 30 temporarily reshapes the vessel at the deployment site.

Once the expandable element 30 has reached an expanded configuration corresponding to a temporary partial remodeling of the vessel at the deployment site, the effective diameter of the vessel may be obtained by measuring the current diameter of the expandable element 30. In some cases, this may be done using fluoroscopy to visualize the expandable element 30 itself. Fluoroscopy allows measurements to be taken of features and elements that are visualized during fluoroscopy. In some cases, instead of measuring the current diameter of the expandable element 30, it may be easier to determine a current length of the expandable element 30. It will be appreciated that for some expandable elements 30 such as braids, there may be known relationships between changes in length of the braid and corresponding changes in diameter of the braid. In some cases, it may be feasible to determine a change in braid angle of the braid. There are known relationships between changes in braid angle, changes in diameter and changes in length. Once the effective diameter has been directly measured or indirectly inferred, the expandable element 30 may be collapsed into its collapsed configuration by advancing the inner shaft 26 distally, relative to the outer shaft 28, in the direction indicated by the arrow 36 to collapse the expandable element 30 into the collapsed configuration for withdrawal or for moving to another measurement site.

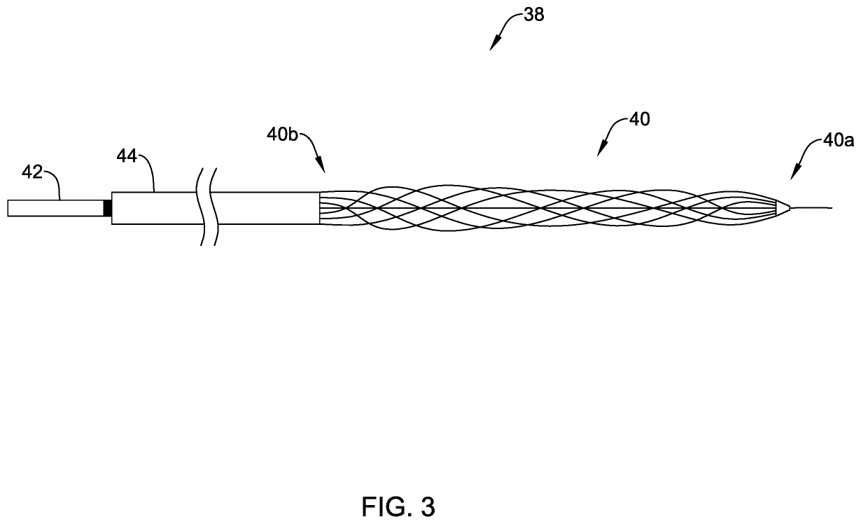

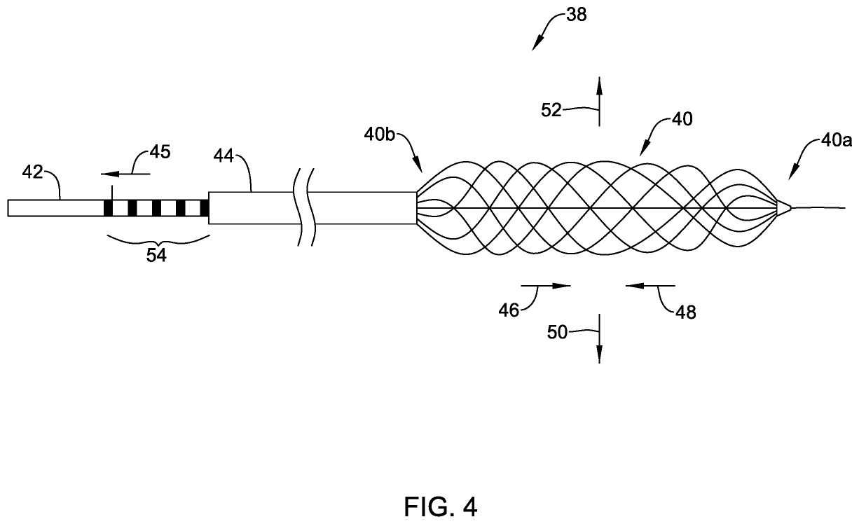

FIG. 3 is a schematic view of an illustrative apparatus 38 having an expandable element 40 shown in a collapsed configuration and FIG. 4 is a schematic view of the illustrative apparatus 38 with the expandable element 40 shown in an expanded configuration. The apparatus 38 may be used in determining an effective vessel diameter. The apparatus 38 includes an inner shaft 42 and an outer shaft 44. The inner shaft 42 is movable relative to the outer shaft 44. Similar to that shown in FIG. 2, a distal end 40a of the expandable element 38 is secured to the inner shaft 42 and a proximal end 40b of the expandable element 38 is secured to the outer shaft 44. In some cases, the expandable element 38 may be a braided structure. Consequently, moving the inner shaft 42 proximally, in a direction indicated by an arrow 44, causes the expandable element 40 to decrease in length, as indicated by arrows 46 and 48, and correspondingly increase in diameter, as indicated by arrows 50 and 52.

In some cases, fluoroscopy may be used to directly measure or indirectly infer one or more dimensions relating to the expandable element 40. However, in some cases, the inner shaft 42 may include markings 54 that function as an indication of how far the inner shaft 42 has moved relative to the outer shaft 44. This distance corresponds to a change in length of the expandable element 40. Because there are known relationships between changes in length and corresponding changes in diameter of the expandable element 40, particularly if the expandable element 40 is a braided structure, the markings 54 may include effective diameter indications and/or markings indicating changes in length of the expandable element 40. In this case, no fluoroscopy is needed. FIG. 5 shows the apparatus 38 with the expandable element 40 in a more fully expanded configuration. As indicated, the diameter “d” is a function of the length “l” of the expandable element 40.

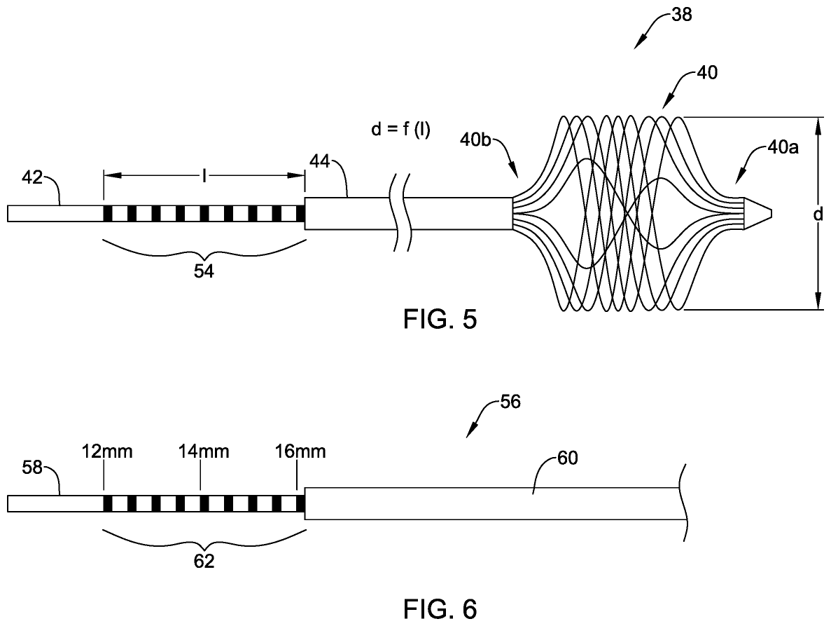

FIG. 6 is a schematic view showing a proximal portion of an illustrative apparatus 56. While not shown, the apparatus 56 includes an expandable element that may be moved between a collapsed configuration and an expanded configuration via relative movement between an inner shaft 58 and an outer shaft 60. The inner shaft 58 includes a scale 62 that shows a numerical conversion between change in length of the expandable element and a corresponding diameter of the expandable element. As shown, the scale 62 may for example include markings for 12 mm, 14 mm and 16 mm. These are merely illustrative, as the apparatus 56 may be adapted for taking measurements in any size blood vessel.

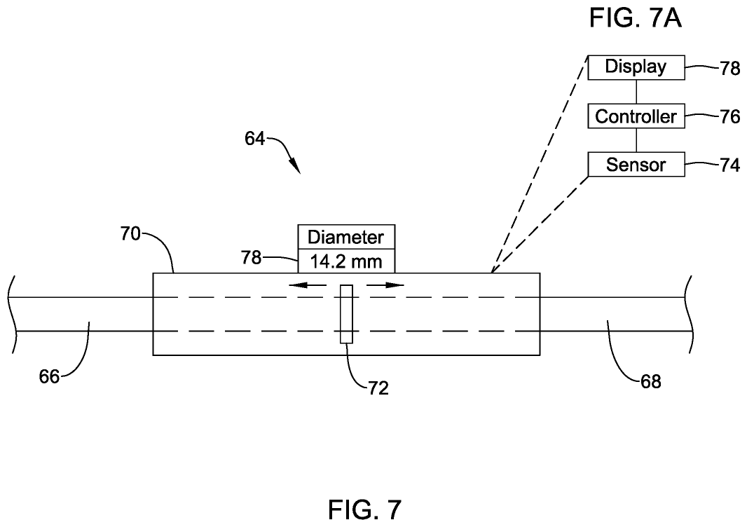

FIG. 7 is a schematic view showing a proximal portion of an illustrative apparatus 64. While not shown, the apparatus 64 includes an expandable element that may be moved between a collapsed configuration and an expanded configuration via relative movement between an inner shaft 66 and an outer shaft 68. The apparatus 64 includes a size indicator 70 that includes a position element 72 that moves relative to the size indicator 70 as the inner shaft 66 moves relative to the outer shaft 68. As seen in FIG. 7A, a sensor 74 is adapted to sense movement of the position element 72 and reports that movement to a controller 76. The controller 76 converts the detected movement of the position element 72 into a corresponding diameter, and displays the diameter on a display 78.

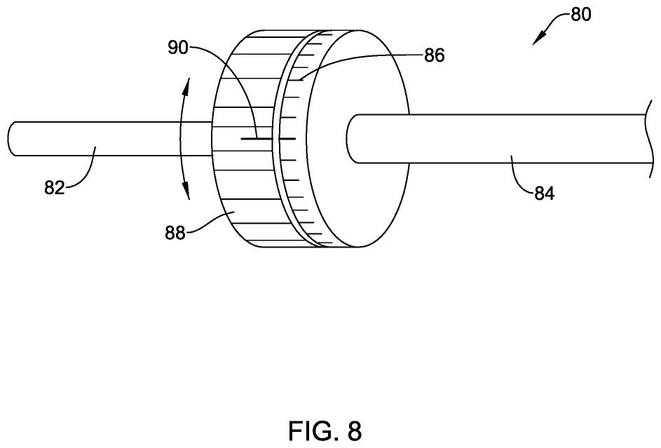

FIG. 8 is a schematic view showing a proximal portion of an illustrative apparatus 80. While not shown, the apparatus 80 includes an expandable element that may be moved between a collapsed configuration via relative movement between an inner shaft 82 and an outer shaft 84. The unseen expandable element may be similar to the expandable element 18 (FIG. 1), the expandable element 30 (FIG. 2) or the expandable element 40, for example. The apparatus 80 includes an indicator 86 that is secured relative to the outer shaft 84. In some cases, the indicator 86 may include an alphanumerical scale that provides an indication of dimension. For example, the alphanumerical scale may include a series of markings “5 mm”, “6 mm”, “7 mm” and so on, particularly if indicating a diameter of the expandable element or perhaps a series of markings “20 mm”, “21 mm”, “22 mm” and so on, particularly if indicating a length of the expandable element. It will be appreciated that these numerical examples are illustrative only and are not intended to be limiting.

In some cases, the indicator 86 may include an alphanumerical scale that provides an indication of a change in diameter of the expandable element. In some cases, the indicator 86 may include an alphanumerical scale that provides an indication of a change in length of the expandable element. A rotary driver 88 is coupled with the inner shaft 82 such that rotation of the rotary driver 88 causes translation of the inner shaft 82 relative to the outer shaft 84. In some cases, the rotary driver 88 may include a knurled or otherwise textured surface to facilitate grasping and turning the rotary driver 88. The rotary driver 88 may include a screw drive, for example. The rotary driver 88 includes an indicator point 90 that may be adapted to point to a particular spot on the indicator 86. As the rotary driver 88 rotates, and the expandable element correspondingly changes in length and diameter, the indicator point 90 will point to a particular spot on the indicator 86. When the indicator 86 includes an alphanumeric scale (schematically shown as a series of spaced apart lines), the user will be able to easily read a spot on the indicator 86 that the indicator point 90 is pointing to, thereby easily reading a numerical value for a length or a diameter of the unseen expandable element.

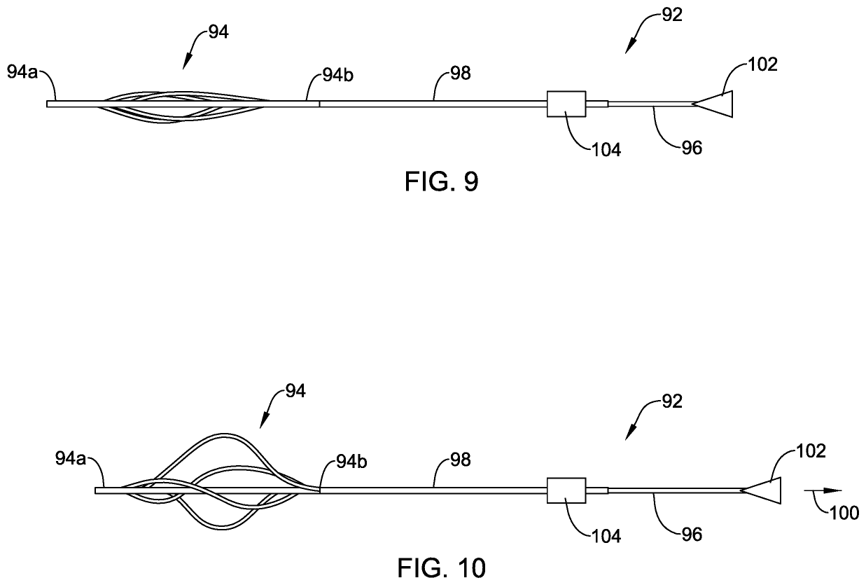

FIG. 9 is a schematic view of an illustrative apparatus 92 that includes an expandable element 94, with the expandable element 94 shown in a collapsed configuration. FIG. 10 shows the illustrative apparatus 92 with the expandable element 94 shown in an expanded configuration. In some cases, the expandable element 94 may be a spiral cut hypotube that is adapted to expand in diameter in response to a decrease in length. The apparatus 92 includes an inner shaft 96 and an outer shaft 98. The expandable element 94 has a distal end 94a that is secured to the inner shaft 96 and a proximal end 94b that is secured to the outer shaft 98. As a result, the expandable element 94 may be moved from its collapsed configuration (shown in FIG. 9) to its expanded configuration (shown in FIG. 10) by withdrawing the inner shaft 96 (relative to the outer shaft 98) in a direction indicated by an arrow 100. In some cases, the expandable element 94 may include two or more lobes. In some cases, the inner shaft 96 includes a handle 102 to facilitate withdrawing the inner shaft 96. In some cases, the apparatus 92 may include a block 104 that is secured relative to the outer shaft 98, and that may be pinned down in order to allow for one handed operation. Fluoroscopy may be used to ascertain the effective vessel diameter.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super clastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), cthylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of determining an effective vessel diameter at a deployment site within a vessel using a measurement apparatus, the measurement apparatus including an expandable element that is moveable between a collapsed configuration for advancement and an expanded configuration for measurement, the method comprising:
advancing the measurement apparatus to the deployment site with the expandable element in its collapsed configuration;
causing the expandable element to expand from its collapsed configuration to its expanded configuration;
obtaining one or more measurements pertaining to the expandable element in its expanded configuration; and
using the obtained one or more measurements to determine an effective vessel diameter at the deployment site.

2. The method of claim 1, wherein the expandable element is adapted to limit its expanded configuration when the expandable element apposes an interior wall of the vessel.

3. The method of claim 1, wherein the measurement apparatus comprises an outer sheath adapted to extend over the expandable element, and causing the expandable element to expand from its collapsed configuration to its expanded configuration comprises withdrawing the outer sheath proximally to uncover the expandable element.

4. The method of claim 3, wherein the expandable element is adapted to regain its expanded configuration when no longer covered by the outer sheath.

5. The method of claim 1, wherein the measurement apparatus comprises:
an outer member secured to a proximal region of the expandable element; and
an inner member extending through an interior of the outer member, the inner member secured to a distal region of the expandable element;
wherein causing the expandable element to expand from its collapsed configuration to its expanded configuration comprises withdrawing the inner member proximally in order to shorten a length of the expandable element, thereby causing the expandable element to move towards its expanded configuration.

6. The method of claim 1, wherein obtaining one or more measurements comprises using fluoroscopy to determine a diameter of the expandable element in its expanded configuration.

7. The method of claim 1, wherein obtaining one or more measurements comprises determining one or more changes for the expandable element in moving from its collapsed configuration to its expanded configuration.

8. An apparatus for determining an effective diameter of a vessel, the apparatus comprising:
an expandable element having a proximal region and a distal region, the expandable element moveable between a collapsed configuration in which the expandable element has a collapsed length and a collapsed diameter and an expanded configuration in which the expandable element has a decreased length and an increased diameter;
an outer elongate member having a distal region secured relative to the proximal region of the expandable element, the outer elongate member defining a lumen extending therewithin; and
an inner elongate member having a distal region secured relative to the distal region of the expandable element, the inner elongate member extending through the lumen;
wherein proximal movement of the inner elongate member relative to the outer elongate member causes the expandable element to decrease in length and thus increase in diameter until the braid urges the vessel into a more uniform cross-sectional shape; and
wherein the expandable element is adapted to be visible under an imaging technique such that one or more dimensions of the expandable element are measurable.

9. The apparatus of claim 8, wherein the one or more dimensions of the expandable element comprise a diameter of the expandable element.

10. The apparatus of claim 8, wherein the one or more dimensions of the expandable element comprises a change in length of the expandable element.

11. The apparatus of claim 8, further comprising an indicator that provides a diameter of the expandable element.

12. The apparatus of claim 11, wherein the indicator comprises a plurality of markings disposed along the inner elongate member.

13. The apparatus of claim 11, wherein the indicator comprises a rotary dial secured relative to the apparatus, the rotary dial coupled with a screw drive that causes rotation of the rotary dial to cause translation of the inner elongate member relative to the outer elongate member; and wherein the indicator comprises an alphanumeric scale that shows a diameter or a length of the expandable element.

14. The apparatus of claim 8, wherein the expandable element comprises a spiral cut hypotube.

15. The apparatus of claim 8, wherein the expandable element comprises a braid.

16. The apparatus of claim 15, wherein the one or more dimensions of the expandable element comprises a change in braid angle.

17. An apparatus for determining an effective diameter of a vessel at a deployment site, the vessel including vessel walls, the apparatus comprising:
a braid having a proximal region and a distal region, the braid moveable between a collapsed configuration and an expanded configuration, the braid adapted to be visible under fluoroscopy;
an outer sheath overlying the braid, the outer sheath constraining the braid in its collapsed configuration while the outer sheath overlies the braid;
wherein the outer sheath is adapted to be withdrawn proximally to expose the braid, thereby allowing the braid to expand towards its expanded configuration until braid apposes the vessel walls, the braid expansion limited by interaction with the vessel walls; and
wherein fluoroscopy may be used to ascertain one or more dimensions of the braid.

18. The apparatus of claim 17, wherein the one or more dimensions of the braid include a diameter of the braid.

19. The apparatus of claim 17, wherein the one or more dimensions of the braid include a change in length of the braid.

20. The apparatus of claim 17, wherein the one or more dimensions of the braid comprises a change in braid angle.
